(19) United States
(12) Patent Application Publication
(10) Pub. No.: US 2022/0409368 A1
(43) Pub. Date: Dec. 29, 2022

(54) PROSTHETIC HEART VALVE LEAFLET ASSEMBLIES AND METHODS

(71) Applicant: Edwards Lifesciences Corporation, Irvine, CA (US)

(72) Inventors: Elena Sherman, Pardes Hana (IL); Noam Nir, Pardes-Hanna (IL)

(21) Appl. No.: 17/901,662

(22) Filed: Sep. 1, 2022

Related U.S. Application Data
(63) Continuation of application No. PCT/US2021/020206, filed on Mar. 1, 2021.
(60) Provisional application No. 62/985,558, filed on Mar. 5, 2020.

(51) Int. Cl. A61F 2/24

(52) U.S. Cl. CPC A61F 2/2418; A61F 2220/0075

(57) ABSTRACT

An assembly and associated method for a commissure of a prosthetic heart valve is disclosed. As one example, a method includes forming a plurality of commissures with the plurality of leaflets, wherein each commissure is formed by pairing a first commissure tab of a first leaflet with an adjacent, second commissure tab of a second leaflet, and coupling a support strip to the first and second commissure tabs via sutures forming stitches along the support strip, and, for each commissure, securing the commissure to a respective support of a frame by forming one or more suture loops around an external surface of the support strip and the respective support of the frame, the suture loops being formed by end portion extensions of the sutures.

### CROSS REFERENCE TO RELATED APPLICATIONS

This application is a continuation of PCT Application No. PCT/US2021/020206, entitled “PROSTHETIC HEART VALVE LEAFLET ASSEMBLIES AND METHODS,” filed Mar. 1, 2021, which claims the benefit of U.S. Provisional Application No. 62/985,558, entitled “PROSTHETIC HEART VALVE LEAFLET ASSEMBLIES AND METHODS,” filed on Mar. 5, 2020, wherein each of the above-referenced applications is incorporated by reference herein in its entirety.

### FIELD

The present disclosure relates to prosthetic heart valves, and to methods and assemblies for forming commissures associated with leaflets of such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Prosthetic valves that rely on a mechanical actuator for expansion can be referred to as “mechanically expandable” prosthetic heart valves. The actuator typically takes the form of pull cables, sutures, wires and/or shafts that are configured to transmit expansion forces from a handle of the delivery apparatus to the prosthetic valve. Most expandable, transcatheter heart valves comprise a cylindrical metal frame or stent and prosthetic leaflets mounted inside the frame. The leaflets may be attached to the frame at commissure tabs of the leaflets. However, in some configurations, forces experienced during operation of the prosthetic valve and/or other conditions may be concentrated at the commissure tabs, which may compromise the structure of the leaflets and/or cause the leaflets to detach from the frame. Furthermore, the attachment of the commissure tabs to the frame in such configurations may require delicate assembly skills to ensure proper attachment and reduce damage to the leaflets. Elements used to attach the commissure tabs to the frame may also obscure an attachment region of the delivery apparatus to the frame, thereby increasing a difficulty in achieving proper attachment of the delivery apparatus.

Accordingly, a need exists for improved prosthetic heart valve leaflet assemblies and methods for assembling the leaflet assemblies to a frame of the prosthetic heart valve.

### SUMMARY

Described herein are embodiments of methods for assembling a prosthetic heart valve, methods of assembling a commissure of a prosthetic valve, and a prosthetic heart valve including a plurality of commissures. In some embodiments, the commissures may be formed by coupling a pair of adjacent commissure tabs of adjacent leaflets of the prosthetic heart valve. The commissures may include a support strip that includes suture lines formed of apertures, through which one or more sutures are respectively passed to form stitches coupling the support strip to the commissure tabs. In some embodiments, each commissure is secured to a support structure of a frame of the prosthetic heart valve by one or more suture loops that are formed by end portion extensions of the sutures that extend around an external surface of the support strip and the support structure.

In one representative embodiment, a method of assembling a prosthetic heart valve comprising a plurality of leaflets can include: forming a plurality of commissures with the plurality of leaflets, wherein each commissure is formed by pairing a first commissure tab of a first leaflet with an adjacent, second commissure tab of a second leaflet, and coupling a support strip to the first and second commissure tabs via sutures extending through apertures of suture lines that are disposed on opposing sides of the support strip, the sutures forming stitches along the support strip, and, for each commissure, securing the commissure to a respective support of a frame by forming one or more suture loops around an external surface of the support strip and the respective support of the frame, wherein the one or more suture loops are formed by end portion extensions of the sutures.

In another representative embodiment, a prosthetic heart valve can include: an annular frame comprising a plurality of commissure support portions, and a plurality of leaflets, each leaflet having a commissure tab that is coupled to an adjacent commissure tab of another leaflet via a support strip to form a commissure of an associated commissure tab pair, where, for each commissure tab pair, sutures extend through apertures of suture lines that are disposed on opposing sides of the support strip, the sutures forming stitches along the support strip coupling the support strip to each commissure tab of the commissure tab pair, and where each commissure is secured to a corresponding respective commissure support portion of the plurality of commissure support portions by one or more suture loops that are formed by end portion extensions of the sutures that extend around an external surface of the support strip and the respective commissure support portion of the annular frame.

In yet another representative embodiment, a method of assembling a prosthetic heart valve comprising a plurality of leaflets includes: forming a plurality of commissures with the plurality of leaflets, wherein each commissure is formed by pairing a first commissure tab of a first leaflet with an adjacent, second commissure tab of a second leaflet, coupling a support strip to the first and second commissure tabs via primary sutures extending through the support strip and the first and second commissure tabs to form stitches along primary suture lines of the support strip, and coupling end portions of the support strip to the first and second commissure tabs, respectively, via secondary sutures extending through the support strip and the first and second commissure tabs, respectively, to form stitches along respective secondary suture lines of the support strip, and, for each commissure, securing the commissure to a respective support portion of a frame of the prosthetic heart valve by one or more suture loops that are formed by end portion extensions of the secondary sutures that extend around an external surface of the support strip and the respective commissure support portion of the annular frame.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an exemplary embodiment of a prosthetic heart valve.

FIG. 2 illustrates a cross-sectional view of an example commissure of a prosthetic valve, in which the commissure is secured to a support post of the prosthetic valve.

FIGS. 3A and 3B illustrate different views of an example commissure secured to a support structure using suture loops that extend around an outer surface of a support strip of the commissure.

FIGS. 4A-4F illustrate different example stages for securing an assembled commissure to a support structure using suture loops.

FIG. 5 is a flow chart of an example method for assembling a commissure and securing the commissure to a support structure of a frame of a prosthetic valve.

FIG. 6 is a side elevation view of a delivery apparatus for a prosthetic heart valve, according to one example.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved. The technologies from any example can be combined with the technologies described in any one or more of the other examples. In view of the many possible embodiments to which the principles of the disclosed technology may be applied, it should be recognized that the illustrated embodiments are only preferred examples and should not be taken as limiting the scope of the disclosed technology.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein, with reference to the prosthetic heart valve and the transcatheter delivery system, “proximal” refers to a position, direction, or portion of a component that is closer to the user and a handle of the delivery system that is outside the patient, while “distal” refers to a position, direction, or portion of a component that is further away from the user and the handle and closer to the implantation site. The terms “longitudinal” and “axial” refer to an axis extending in the proximal and distal directions, unless otherwise expressly defined.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Examples of the Disclosed Technology

Described herein are examples of prosthetic heart valves, commissures for prosthetic valves, and methods for assembling commissures of prosthetic valves. The prosthetic heart valves may include a frame and a plurality of leaflets attached to the frame via commissures formed by joining pairs of adjacent ends of the leaflets. The formation of the commissures may include attaching a support strip to the leaflets (e.g., to commissure tabs of the leaflets) using sutures that forms stitches along suture lines. Opposing ends of the support strip may be joined together, directly or indirectly (e.g., via one or more sutures), and the opposing ends of the support strip and/or sutures that join the opposing ends of the support strip may be wrapped around a corresponding commissure support portion of the frame of the prosthetic valve. Additional suture loops may be formed by wrapping end portion extensions of sutures used in assembling the commissure around an external surface of the support strip and the support portion of the frame. In this way, forces experienced by the leaflets during radial expansion and compression of the frame and/or as the leaflets open and closed during operation of the prosthetic valve may be at least partially absorbed by the support strip and distributed across the additional suture loops, reducing the stresses exerted on the leaflets and increasing a strength of the coupling of the commissure to the support portion of the frame. As the support strip may be made of more robust material than the leaflets, the overall strength of the commissure may be increased relative to other configurations. Furthermore, the additional suture loops may further tighten the coupling of the commissure to the support portion of the frame (e.g., increasing forces holding the commissure to the support portion), thereby reducing movement of the commissure around the support portion to which it is coupled. As the above-described relative motion otherwise causes abrasion and damage to the commissure components, reducing such motion increases the overall durability of the commissure assembly.

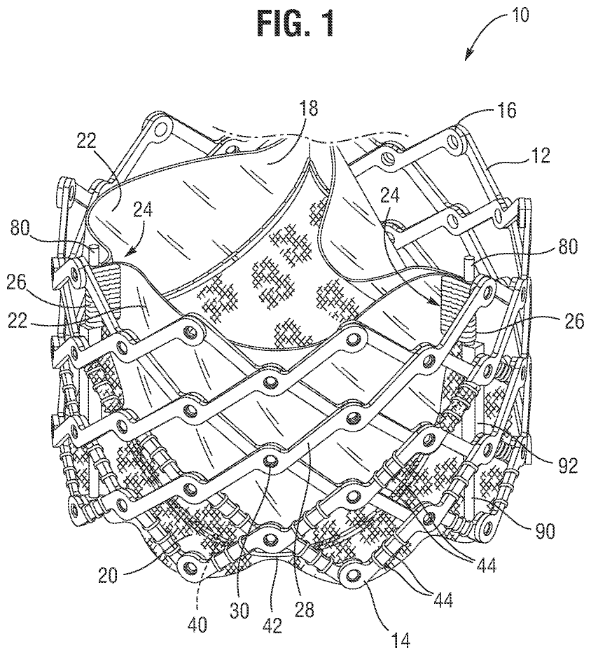

FIG. 1 shows an exemplary prosthetic heart valve 10, according to one embodiment. The prosthetic heart valve 10 can be radially compressible and expandable between a radially compressed configuration for delivery into a patient and a radially expanded configuration. In particular embodiments, the prosthetic heart valve 10 can be implanted within the native aortic valve, although it also can be implanted at other locations in the heart, including within the native mitral valve, the native pulmonary valve, and the native tricuspid valve. The prosthetic heart valve 10 can include an annular stent or frame 12 having a first end 14 and a second end 16.

In the depicted embodiment, the first end 14 is an inflow end and the second end 16 is an outflow end. The outflow end 16 can be coupled to a delivery apparatus for delivering and implanting the prosthetic heart valve within the native aortic valve is a transfemoral, retrograde delivery approach. Thus, in the delivery configuration of the prosthetic heart valve, the outflow end 16 is the proximal-most end of the prosthetic valve. In other embodiments, the inflow end 14 can be coupled to the delivery apparatus, depending on the particular native valve being replaced and the delivery technique that is used (e.g., trans-septal, transapical, etc.). For example, the inflow end 14 can be coupled to the delivery apparatus (and therefore is the proximal-most end of the prosthetic heart valve in the delivery configuration) when delivering the prosthetic heart valve to the native mitral valve via a trans-septal delivery approach.

The frame 12 can be made of any of various suitable materials, such as stainless steel, a cobalt chromium alloy, or a nickel titanium alloy (“NiTi”), for example Nitinol. Referring again to FIG. 1, as shown, the frame 12 can include a plurality of interconnected struts 28 arranged in a lattice-type pattern. The struts 28 are shown as positioned diagonally, or offset at an angle relative to, and radially offset from, a longitudinal axis of the prosthetic heart valve 10 when the prosthetic heart valve 10 is in the expanded configuration. In other implementations, the struts 28 can be offset by a different amount than depicted in FIG. 1, or some or all of the struts 28 can be positioned parallel to the longitudinal axis of the prosthetic heart valve 10.

In the illustrated embodiment, the struts 28 are pivotably coupled to one another at one or more pivot joints along the length of each strut. For example, in the illustrated configuration, each of the struts 28 can be formed with apertures at opposing ends of the strut and apertures spaced along the length of the strut. Respective hinges can be formed at the locations where struts 28 overlap each other via fasteners or pivot members, such as rivets or pins 30 that extend through the apertures. The hinges can allow the struts 28 to pivot relative to one another as the frame 12 is radially expanded or compressed, such as during assembly, preparation, or implantation of the prosthetic heart valve 10.

In some embodiments, the frame 12 can be constructed by forming individual components (e.g., the struts and fasteners of the frame) and then mechanically assembling and connecting the individual components together. In other embodiments, the struts 28 are not coupled to each other with respective hinges but are otherwise pivotable or bendable relative to each other to permit radial expansion and contraction of the frame 12. For example, the frame 12 can be formed (e.g., via laser cutting, electroforming or physical vapor deposition) from a single piece of material (e.g., a metal tube). Further details regarding the construction of the frame and the prosthetic heart valve are described in U.S. Patent Application Publication Nos. 2018/0153689, 2018/0344456, and 2019/0060057, all of which are incorporated herein by reference.

The prosthetic heart valve 10 can also include a valvular structure 18 which is coupled to the frame 12 and configured to regulate the flow of blood through the prosthetic heart valve 10 from the inflow end 14 to the outflow end 16. The prosthetic heart valve 10 can further include a plurality of actuators 80 mounted to and equally spaced around the inner surface of the frame 12. The actuators are configured to apply expansion and compression to the frame for radially expanding and compressing the prosthetic valve.

In the illustrated embodiment, the actuators 80 are linear actuators, each of which comprises an inner member, or piston, 90 and an outer member, or cylinder, 92. The inner member 90 is pivotably coupled to a junction of the frame, such as at the first end 14, while the outer member 92 is pivotably coupled to another junction of the frame closer to the second end 16. Moving the inner member 90 proximally relative to the outer member 92 and/or moving the outer member 92 distally relative to the inner member 90 is effective to radially expand the prosthetic valve. Conversely, moving the inner member 90 distally relative to the outer member 92 and/or moving the outer member 92 proximally relative to the inner member 90 is effective to radially compress the prosthetic valve. The actuators 80 can include locking mechanisms that are configured to retain the prosthetic valve in an expanded state inside the patient's body.

In some embodiments, each of the actuators 80 can be configured to form a releasable connection with one or more respective actuators of a delivery apparatus of a transcatheter delivery system. The actuators of the delivery apparatus can transmit forces from a handle of the delivery apparatus to the actuators 80 for expanding or compressing the prosthetic valve. Further details of the actuators, locking mechanisms and delivery apparatuses for actuating the actuators can be found in U.S. Patent Application Publication Nos. 2018/0153689, 2019/0060057 and 2018/0325665, each of which is incorporated herein by reference in its entirety. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves discloses herein.

In some embodiments, each of the actuators 80 can be used to support a respective commissure 24 (described below). As such, the actuators 80 can include commissure support portions for supporting and attaching commissures 24 of the valvular structure 18 to the frame 12, as described further herein.

The valvular structure 18 can include, for example, a leaflet assembly comprising one or more leaflets 22 (three leaflets 22 in the illustrated embodiment) made of a flexible material. The leaflets 22 of the leaflet assembly can be made from in whole or part, biological material, bio-compatible synthetic materials, or other such materials. Suitable biological material can include, for example, bovine pericardium (or pericardium from other sources). The leaflets 22 can be arranged to form commissures 24, which can be, for example, mounted to commissure support portions of respective actuators 80. Further details regarding transcatheter prosthetic heart valves, including the manner in which the valvular structure can be coupled to the frame 12 of the prosthetic heart valve 10, can be found, for example, in U.S. Pat. Nos. 6,730,118, 7,393,360, 7,510,575, 7,993,394, and 8,652,202, and U.S. Patent Application Publication No. 2018/0325665, all of which are incorporated herein by reference in their entireties.

In some embodiments, as shown in FIG. 1, the commissures 24 can be mounted (e.g., sutured) directly to commissure support portions of the actuators 80 of the frame 12 via commissure attachments 26, which can be strips of fabric. As one example, each commissure attachment 26 may be wrapped around a corresponding actuator 80 and a pair of adjacent commissure tabs of adjacent leaflets and secured to the commissure tabs and the actuator via one or more stitches extending through the commissure attachment and the pair of the commissure tabs. In other embodiments, the commissures 24 can be mounted to support struts or posts of the frame that are separate from the actuators 80.

The prosthetic heart valve 10 can also include one or more skirts or sealing members. For example, as shown in FIG. 1, the prosthetic heart valve 10 can include an inner skirt 20 mounted on the inner surface of the frame 12. As shown in FIG. 1, the inner skirt 20 is a circumferential inner skirt that spans an entire circumference of the inner surface of the frame 12. The inner skirt 20 can function as a sealing member to prevent or decrease paravalvular leakage (e.g., when the valve is placed at the implantation site) and as an attachment surface to anchor the leaflets 22 to the frame 12.

For example, as shown, a cusp edge portion 40 of each leaflet 22 (the inflow edge portion) can be secured to the inner skirt 20 with stitching 42 (referred to as a “scallop line”). The upper and lower edge portions of the inner skirt 20 can be secured to the frame with suture loops 44 that extending through the inner skirt and around adjacent struts 28 of the frame. In this manner, the cusp edge portions of the leaflets are supported by the inner skirt 20 and the commissures are supported by actuators 80.

The prosthetic heart valve 10 can also include an outer skirt mounted on the outer surface of the frame 12 (not shown in FIG. 1). The outer skirt can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials (e.g., PET) or natural tissue (e.g., pericardial tissue). The inner and outer skirts can be mounted to the frame using sutures, an adhesive, welding, and/or other means for attaching the skirts to the frame.

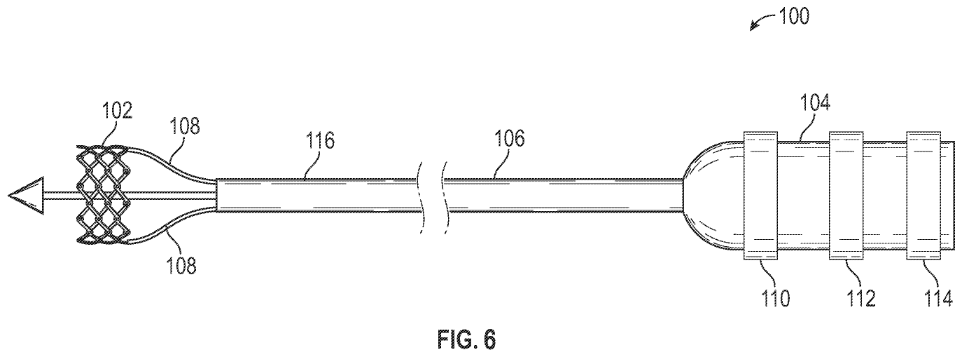

FIG. 6 illustrates a delivery apparatus 100, according to one embodiment, adapted to deliver a prosthetic heart valve 102, such as the illustrated prosthetic heart valve 10, described above with respect to FIG. 1. The prosthetic valve 102 can be releasably coupled to the delivery apparatus 100. It should be understood that the delivery apparatus 100 and other delivery apparatuses disclosed herein can be used to implant prosthetic devices other than prosthetic valves, such as stents or grafts.

The delivery apparatus 100 in the illustrated embodiment generally includes a handle 104, a first elongated shaft 106 (which comprises an outer shaft in the illustrated embodiment) extending distally from the handle 104, at least one actuator assembly 108 extending distally through the outer shaft 106. The at least one actuator assembly 108 can be configured to radially expand and/or radially collapse the prosthetic valve 102 when actuated.

Though the illustrated embodiment shows two actuator assemblies 108 for purposes of illustration, it should be understood that one actuator 108 can be provided for each actuator on the prosthetic valve. For example, three actuator assemblies 108 can be provided for a prosthetic valve having three actuators. In other embodiments, a greater or fewer number of actuator assemblies can be present.

In some embodiments, a distal end portion 116 of the shaft 106 can be sized to house the prosthetic valve in its radially compressed, delivery state during delivery of the prosthetic valve through the patient's vasculature. In this manner, the distal end portion 116 functions as a delivery sheath or capsule for the prosthetic valve during delivery,

The actuator assemblies 108 can be releasably coupled to the prosthetic valve 102. For example, in the illustrated embodiment, each actuator assembly 108 can be coupled to a respective actuator of the prosthetic valve 102. Each actuator assembly 108 can comprise a support tube, an actuator member, and a locking tool. When actuated, the actuator assembly can transmit pushing and/or pulling forces to portions of the prosthetic valve to radially expand and collapse the prosthetic valve as previously described. The actuator assemblies 108 can be at least partially disposed radially within, and extend axially through, one or more lumens of the outer shaft 106. For example, the actuator assemblies 108 can extend through a central lumen of the shaft 106 or through separate respective lumens formed in the shaft 106.

The handle 104 of the delivery apparatus 100 can include one or more control mechanisms (e.g., knobs or other actuating mechanisms) for controlling different components of the delivery apparatus 100 in order to expand and/or deploy the prosthetic valve 102. For example, in the illustrated embodiment the handle 104 comprises first, second, and third knobs 110, 112, and 114.

The first knob 110 can be a rotatable knob configured to produce axial movement of the outer shaft 106 relative to the prosthetic valve 102 in the distal and/or proximal directions in order to deploy the prosthetic valve from the delivery sheath 116 once the prosthetic valve has been advanced to a location at or adjacent the desired implantation location with the patient's body. For example, rotation of the first knob 110 in a first direction (e.g., clockwise) can retract the sheath 116 proximally relative to the prosthetic valve 102 and rotation of the first knob 110 in a second direction (e.g., counter-clockwise) can advance the sheath 116 distally. In other embodiments, the first knob 110 can be actuated by sliding or moving the knob 110 axially, such as pulling and/or pushing the knob. In other embodiments, actuation of the first knob 110 (rotation or sliding movement of the knob 110) can produce axial movement of the actuator assemblies 108 (and therefore the prosthetic valve 102) relative to the delivery sheath 116 to advance the prosthetic valve distally from the sheath 116.

The second knob 112 can be a rotatable knob configured to produce radial expansion and/or contraction of the prosthetic valve 102. For example, rotation of the second knob 112 can move the actuator member and the support tube axially relative to one another. Rotation of the second knob 112 in a first direction (e.g., clockwise) can radially expand the prosthetic valve 102 and rotation of the second knob 112 in a second direction (e.g., counter-clockwise) can radially collapse the prosthetic valve 102. In other embodiments, the second knob 112 can be actuated by sliding or moving the knob 112 axially, such as pulling and/or pushing the knob.

The third knob 114 can be a rotatable knob configured to retain the prosthetic heart valve 102 in its expanded configuration. For example, the third knob 114 can be operatively connected to a proximal end portion of the locking tool of each actuator assembly 108. Rotation of the third knob in a first direction (e.g., clockwise) can rotate each locking tool to advance the locking nuts to their distal positions to resist radial compression of the frame of the prosthetic valve, as described above. Rotation of the knob 114 in the opposite direction (e.g., counterclockwise) can rotate each locking tool in the opposite direction to decouple each locking tool from the prosthetic valve 102. In other embodiments, the third knob 114 can be actuated by sliding or moving the third knob 114 axially, such as pulling and/or pushing the knob.

Although not shown, the handle 104 can include a fourth rotatable knob operative connected to a proximal end portion of each actuator member. The fourth knob can be configured to rotate each actuator member, upon rotation of the knob, to unscrew each actuator member from the proximal portion of a respective actuator. As described above, once the locking tools and the actuator members are uncoupled from the prosthetic valve 102, they can be removed from the patient.

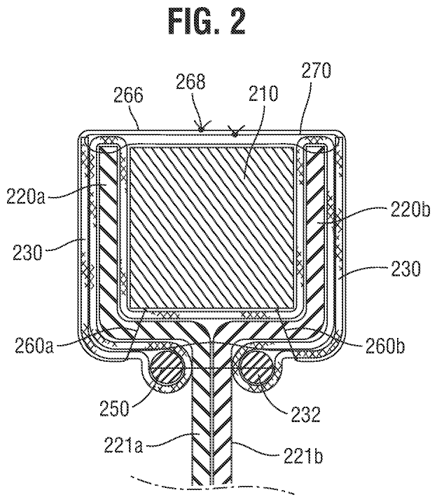

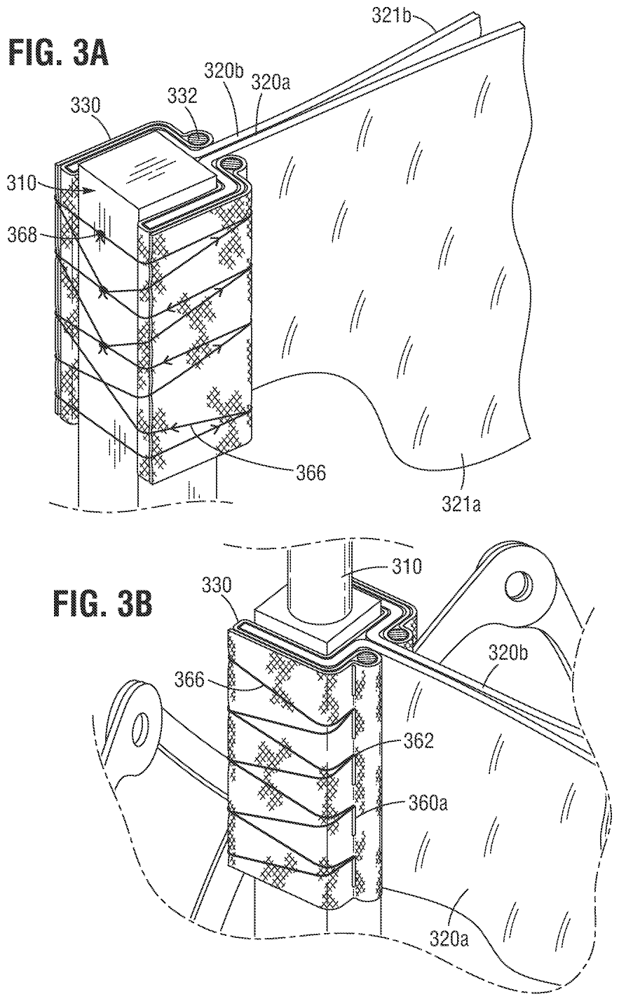

FIGS. 2-3B show example commissure tab assemblies and attachments of commissure tab assemblies to a commissure post or other support structure of a frame of a prosthetic valve. FIG. 2 shows and example cross-sectional view of the commissure tab assembly and FIGS. 3A and 3B show isometric views of an example commissure tab assembly from two different view angles.

A commissure tab assembly may be pre-assembled prior to attachment thereof to the frame by performing a pre-assembly process. The pre-assembly process includes, using the components of FIG. 2 in one example, placing a support strip 230 (e.g., a flexible cloth/fabric) over a pair of adjacent commissure tabs 220a and 220b of respective leaflets 221a and 221b and folding end portions of the support strip over end portions of the commissure tabs.

A reinforcing element 232, described in more detail below, may be placed against a surface of the support strip on each opposing side of the support strip, such that at least a portion of the support strip is sandwiched between the reinforcing element 232 and a respective commissure tab 220a or 220b on each opposing side of the support strip. In order to secure the support strip to the commissure tabs and the reinforcing element, the pre-assembly process may further include extending a primary suture 250 through, in order (or in the reverse order, e.g., starting from the last-listed element and extending through the following list of elements, in reverse, to the first-listed element), a first portion of a reinforcing element 232, a first portion on a first side of a support strip (e.g., a flexible cloth/fabric) 230, a first commissure tab 220a, a second commissure tab 220b (where commissure tabs 220a and 220b are two commissure tabs of adjacent leaflets, e.g., leaflets 221a and 221b), a second portion on a second side of the support strip 230, and a second portion of the reinforcing element 232. The second side of the support strip is opposite the first side relative to a center of the support strip (e.g., a centerline that divides a width/longest dimension of the strip in half).

Although not shown in FIGS. 3A and 3B, it is to be understood that a primary suture may be similarly applied to the commissure assembly shown therein in a similar manner as described above with respect to FIG. 2 (e.g., extending the primary suture through portions of reinforcing elements 332, one or more layers of a support strip 330, and commissure tabs 320a and 320b of leaflets 321a and 321b, respectively).

The support strip (e.g., support strip 230 of FIGS. 2 and/or 330 of FIGS. 3A and 3B) may be a strip of any suitable material (e.g., fabric), which may include material that is stronger (e.g., more resilient to tearing and/or deforming) than a material used for forming the leaflets and/or the commissure tab portions of the leaflets of the prosthetic valve. In one example, the support strip is a polyethylene terephthalate (PET) fabric, although various other suitable biocompatible fabrics can be used.

The support strip may be longer in one dimension than another (e.g., having a width/length that is greater than a height of the strip). The support strip may be continuous (e.g., with no gaps and/or having a substantially uniform distribution of the material forming the support strip, other than optional apertures forming the suture lines described herein) and may be relatively thin (e.g., having a thickness that is substantially smaller than the width and height of the strip and substantially smaller than a width or diameter of the reinforcing elements, discussed in more detail below).

The reinforcing elements, such as reinforcing element 232 of FIG. 2 and reinforcing element 332 of FIGS. 3A and 3B, may include a string, a cord, and/or a relatively thick suture (e.g., an Ethibond suture), which may be substantially wider/thicker and/or have a substantially larger diameter (e.g., at least twice as wide/thick and/or have twice the diameter) than stitching sutures (e.g., primary sutures, secondary sutures, described in more detail below, tertiary sutures, described in more detail below, and/or other sutures of the commissure). The reinforcing elements may be substantially the same height (or slightly shorter, such as 1-5% shorter to provide for machining tolerances and avoid extension of the inner reinforcing element past edges of the strip) as the height of the support strip. In other examples, the reinforcing elements can be a relatively narrow strip(s) of fabric, which can be folded lengthwise one or more times to increase its overall thickness. In still other examples, the reinforcing elements can be a metal wire(s) or a bar(s), such as a rectangular or cylindrical bar(s), formed from a metal and/or a polymer.

The reinforcing elements may include two individual reinforcing elements or two portions of a single reinforcing member that extends along outer surfaces of each of the first and the second commissure tabs. For example, a reinforcing member may be folded into a U-shape configuration to form the reinforcing elements (e.g., the reinforcing elements may be individual elements that are coupled to one another and/or form different sections of a single continuous element/member). In other examples, a reinforcing member may be discontinuous and include the reinforcing elements as discrete or separate elements in a spatially separated configuration (e.g., where a first reinforcing element is spatially separated from a second reinforcing element).

In some examples, the reinforcing elements may be aligned with one or more alignment markings and/or suture/stitching lines of the support strip. In some embodiments, the reinforcing elements are placed against outer surfaces of the commissure tabs, respectively, and opposing end or side portions of the support strip are positioned on an opposing side(s) of the reinforcing elements from the respective commissure tabs. For example, each reinforcing element may be sandwiched between a respective portion of the support strip and a respective one of the commissure tabs. In other embodiments, the support strip is at least partially wrapped or folded around the reinforcing elements to at least partially encase the reinforcing elements. In such examples, the portions of the support strip that at least partially encase the reinforcing elements may be placed against the outer surfaces of the commissure tabs, respectively.

Returning to the formation of the pre-assembled commissure, the primary suture 250 may only pass through a single layer of the support strip 230 on either side of the support strip (e.g., a respective layer of the support strip that is positioned between the reinforcing element 232 and a respective one of the commissure tabs 220a and 220b). In order to increase the strength and force distribution provided by the commissure assembly, end portions of each side of the support strip 230 may be further folded over respective portions of the reinforcing element 232 and positioned alongside the respective end portions of the commissure tabs 220a and 220b to form an additional layer of support strip on an outer region of the commissure assembly. For example, as shown in FIG. 2, a single layer of the support strip 230 may be positioned between the support post 210 and the respective commissure tabs 220a and 220b (e.g., along an inner surface of the commissure tabs), while two layers of the support strip 230 may be provided on an opposing side of the respective commissure tabs 220a and 220b from the support post 210 (e.g., along an outer surface of the commissure tabs).

Additional, secondary sutures 260a and 260b may be used to secure the folded support strip to the commissure tabs as shown in FIG. 2. For example, the secondary sutures 260a and 260b may form a plurality of in-and-out stitches that are extended, each, in order (or in the reverse order): through respective first layers of the support strip 230, through respective second layers of the support strip 230, through respective commissure tabs 220a and 220b and through respective third layers of the support strip 230 in a radial direction. The secondary sutures may extend through the multiple layers of the support strip adjacent a respective reinforcing element that is positioned on that side of the support strip (e.g., around which the associated respective side of the support strip is wrapped). As shown in more detail in FIG. 3B, the in-and-out stitches of an example secondary suture 360a may form a suture line 362 that includes a plurality of apertures and/or markings in at least an outer layer of the support strip through which the secondary suture passes. As used herein, the term “suture line” can also be referred to as a “stitch line.” As further used herein in reference to the support strip, the term “aperture” refers to holes in the support strip used for stitching. In some examples, the holes are formed in the support strip while stitching and/or as a result of the stitching (e.g., the stitching formed by the secondary suture in the above example). In other examples, the holes may include pre-formed holes in the support strip that are made before stitching to provide a guide for the stitching that increases speed, integrity, and/or accuracy of assembly.

The pre-assembled commissure tab assembly, assembled as described in any of the examples above, may be attached to a corresponding commissure post, such as post 210 of FIG. 2 or post 310 of FIGS. 3A and 3B, or other commissure support structure of the frame. The commissure post can be a component of an actuator 80 of the prosthetic valve 10 shown in FIG. 1. For example, the upper portion of outer member 92 (FIG. 1) can serve as the commissure post 210 of FIG. 2 and/or post 310 of FIGS. 3A and 3B. In alternative embodiments, the prosthetic valve 10 can include commissure posts separate from the actuators. The separate commissure posts can be mounted to the inner surface of the frame 12, or can be integral portions of the frame, at locations circumferentially spaced from the actuators.

As shown in FIG. 2, the commissure tab assembly may be attached to the commissure post by extending a tertiary suture 270 across a first side of the commissure support post 210 between two opposing sides of the support strip 230, which are respectively disposed adjacent a second and third side of the commissure support post, the second and third sides being two circumferentially opposite sides of the commissure post 210. For example, the tertiary suture 270 may be used to form shoelace stitches around the post 210 and both ends of the suture 270 may be coupled together (e.g., forming a knot 278 at the top and/or bottom end). For illustrative clarity purposes, a tertiary suture and/or stitches formed by a tertiary suture is not shown with respect to the commissure assembly of FIGS. 3A and 3B, however, it is to be understood that stitches formed by a tertiary suture may be provided therein as described above with respect to FIG. 2 (e.g., passing between two sides of the support strip 330 across a side of the support post 310).

The pre-assembled commissure assembly may be further coupled to a support member of a frame of a prosthetic valve using additional reinforcing stitching, as will be described below. For example, the additional reinforcing stitching may be provided in addition to stitching that is formed using a tertiary suture (e.g., tertiary suture 270). For example, an additional reinforcing suture 266 may be extended around a larger region of the support strip (e.g., support strip 230 of FIG. 2) than the tertiary suture to form reinforcing stitching loops around a support post (e.g., support post 210). As shown in FIG. 2, the additional reinforcing suture 266 may wrap around a larger circumferential area of the commissure assembly (e.g., extending more than 270 degrees, or extending 360 degrees in some examples, around the support post 210 in the illustrated example) than the tertiary suture 270.

As further shown in FIG. 2, the additional reinforcing suture 266 passes through different layers of the commissure assembly components at a region of the commissure assembly that is opposite the first side of the commissure support post 210 (e.g., the side of the support post over which the tertiary suture 270 extends). For example, the additional reinforcing suture 266 may pass through, in order or in reverse order, one or more layers of the support strip 230 on a first side of the support strip, the first and second commissure tabs 220a and 220b, respectively, and one or more layers of the support strip 230 on a second side of the support strip opposite the first side. Ends of the additional reinforcing suture 266 may be tied together to form one or more knots 268 positioned along the first side of the support post 210.

As shown in FIG. 3A, an additional reinforcing suture 366 may form a plurality of reinforcing stitches (which may form one or more corresponding suture loops) crossing over a first (e.g., external) side of the support post 310, where at least one (e.g., each) stitch is secured using a respective knot 368 to tie ends of the additional reinforcing suture together. The illustrations in FIGS. 3A and 3B do not show tertiary sutures or associated stitching for clarity purposes, in order to show the additional reinforcing sutures more clearly. However, it is to be understood that the additional reinforcing suture 366 may be provided in addition to a tertiary suture that is used to form stitches across the support post 310 as described above with respect to tertiary suture 270 FIG. 2 in some examples.

In order to reduce the number of apertures in the support strip, and thereby increase the strength of the support strip (as each aperture may compromise the structural integrity of the support strip), the additional reinforcing suture 266 may pass through at least a portion of the apertures in one or more layers of the support strip through which the secondary sutures 260a and 260b pass through. In this way, the additional reinforcing suture 266 may at least partially pass through apertures included in the suture lines formed by the secondary sutures 260a and 260b. For example, as shown in FIG. 3B, the additional reinforcing suture 366 passes through multiple apertures in the secondary suture line 362. As shown, the suture loops extend substantially perpendicularly relative to the extension of the sutures forming stitches along the support strip in the secondary suture line 362. In this way, the secondary suture line 362 extends substantially perpendicularly (e.g., at between an 80 degree and a 100 degree angle) to the suture loops formed by the additional reinforcing suture 266.

In some examples, the additional reinforcing suture may include end portion extensions of one or more other sutures in the commissure assembly (e.g., end portion extensions of the secondary suture 260a/260b of FIG. 2 and/or secondary suture 360a of FIG. 3B). For example, end portion extensions of one or more sutures may be extended through the secondary suture line 362 and utilized to form additional suture loops around an external surface of the support strip 330 and the support post 310 (e.g., instead of being cut or directly tied off), pressing the support strip and/or adjacent commissure components onto the support post 310. The suture extension may be looped around the support strip and the support post (e.g., crossing over one another by extending in the directions indicated by the illustrated arrows on loops formed by the suture 366) to form a series of suture loops. For example, the suture loops may extend from a first region of an interior side of the support post 310 (e.g., a location of the suture line 362), around a first lateral side of the support post, across an external side of the support post, around a second lateral side of the support post, and to a second region of the interior side of the support post (e.g., a location of a suture line opposite the suture line 362, positioned on an opposite side of the support strip 330 from the suture line 362), where the interior side of the support post is opposite of the external side of the support post and the first and second lateral sides of the support post are opposite of one another and extend between the interior side of the support post and the external side of the support post. In this way, the suture loops and/or the stitches forming the suture loops may extend along at least three sides of the support post. In the illustrated example, the suture loops and/or the stitches forming the suture loops further extend along a portion of a remaining side of the support post, thus, the suture loops and/or stitches forming the suture loops extend more than 270 degrees around a circumference of the support post.

The suture loops may be stitched together at a respective end by a knot 368 or other coupling mechanism (e.g., adhesion, twisting, etc.). Such a configuration may be advantageous, as it may be formed in a reduced time and with reduced efforts relative to other securing mechanisms.

In an alternative approach, the suture extensions may be knotted together at each suture loop (or at a subset of the suture loops, such as every other suture loop, a top half of the suture loops, a bottom half of the suture loops, etc.). Such a configuration may provide improved tightness around the support post, as well as improved durability, relative to configurations with a single knot (or fewer knots), as a tear along one loop does not affect all of the remaining loops in the configuration with multiple knots.

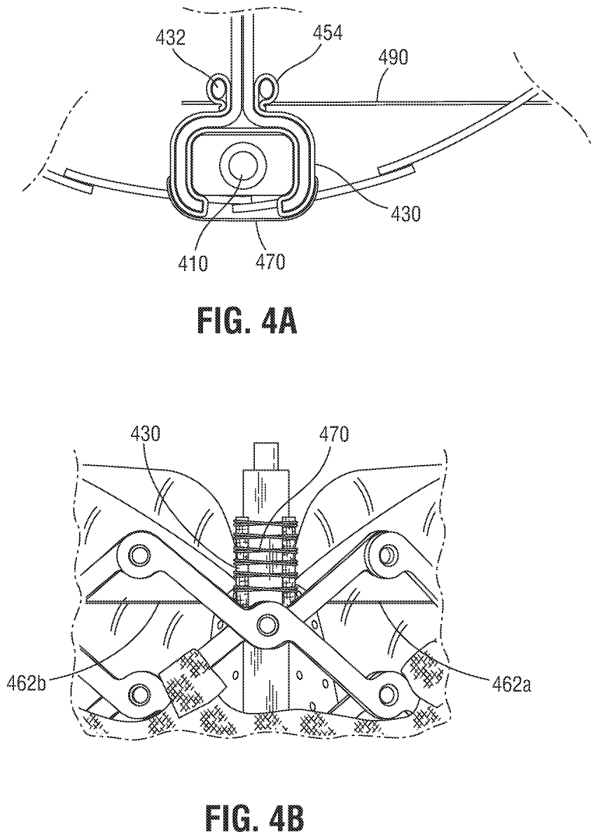

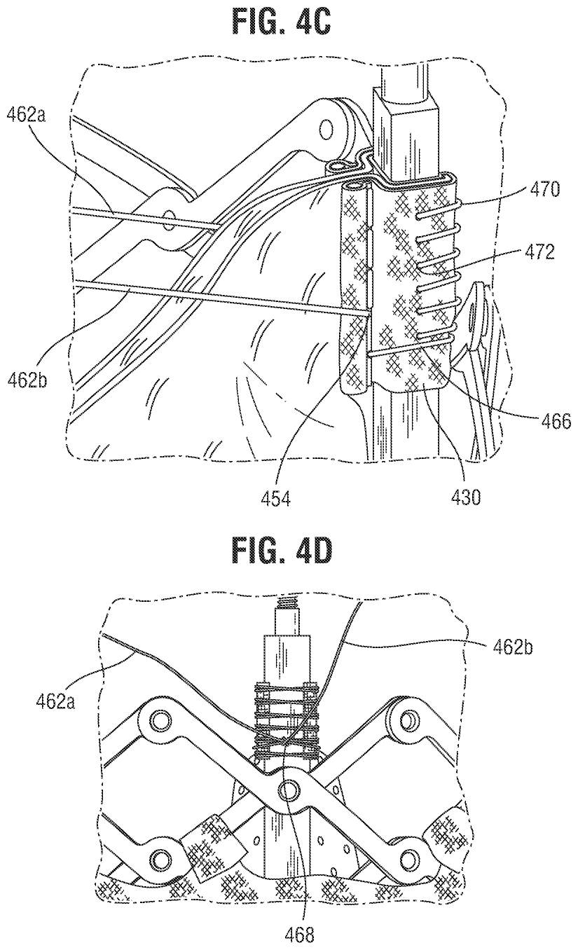

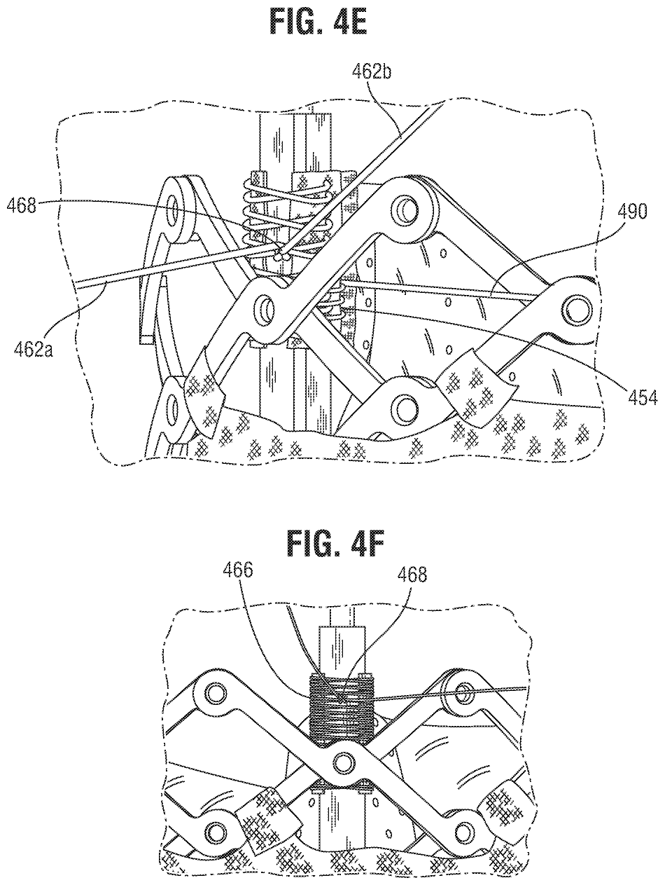

FIGS. 4A-4F show different views and stages for an example approach to forming the above-described loops around a support strip 430 and a support post 410 of a frame of a prosthetic valve in order to secure a commissure (e.g., a pre-assembled commissure as described in any of the above examples) to the support post 410. For example, a pre-assembled commissure, such as the commissure illustrated in FIG. 2 and/or FIGS. 3A and 3B, may be coupled to the support post using tertiary sutures, as described above with respect to tertiary sutures 270 of FIG. 2. In one example, the coupling illustrated in FIG. 2 and/or FIGS. 3A and 3B may be achieved by starting at an upper end of the support post and, using a tertiary suture (e.g., a 5-0 size suture, such as a FORCE FIBER 5-0 suture) with two needles, making stitches (e.g., shoelace stitches) between opposing ends of the support strip to form a series of suture loops (e.g., one of which is shown at 470 in FIG. 4A) across the support post at different locations along a longitudinal axis of the support post. In other examples, the suture loops may be formed from a lower end of the support post working upward along the longitudinal axis. In the example, a first knot (e.g., a single square knot) or other coupling mechanism may be used to secure the commissure (e.g., a knot positioned at a bottommost or topmost end of the commissure, as illustrated at 368 of FIG. 3A). In order to allow for ends of the suture to be used as suture extensions for additional suture loops, the suture may not be cut after forming the knot.

FIG. 4A shows a next stage for further securing and/or tightening a coupling of the commissure to the support post (e.g., after forming the suture loops as described above). The stage shown in FIG. 4A may include making a reinforcement suture by continuing to suture with the suture extensions described above (e.g., the extensions of the tertiary suture). Starting from one end (e.g., a bottom end or a top end) of a secondary suture line (e.g., a bottom or top hole of secondary suture line 362 shown in FIG. 3B), a needle 490 is inserted through the holes of the secondary suture line, a loop is formed over the support post 410, and the needle is again inserted into the same holes. Although described herein as extending through holes of the secondary suture line, it is to be understood that in other examples, the suture extensions may be passed through other suture lines of the commissure or through one or more holes that are not part of other suture lines for the commissure.

As shown in FIGS. 4B and 4C, in a next stage, the suture is placed over the support post 410 and, for a first suture extension 462a, the needle is inserted into a second hole of the secondary suture lines on a first side of the support strip 430. For a second suture extension 462b (e.g., an opposite side of the suture from the first suture extension), the needle is inserted into a second hold of the secondary suture lines on a second side of the support strip. For example, as shown in FIG. 4C, the suture extension 462b passes through the suture line 454.

In a next stage, the suture extensions are wrapped around an outer surface of the support strip 430 and the support post 410 to form additional suture loops, an example of which is shown at 466 in FIG. 4D. The suture extensions are coupled to each other (e.g., via a knot 468), and the suture is not cut (e.g., so that additional suture loops may be formed). This process is continued for further additional suture loops that pass through progressively higher/lower holes in the secondary suture line, as shown in FIGS. 4E and 4F. Accordingly, the suture loops 466 may extend between secondary suture lines 454 on opposing sides of the support strip 430, while tertiary suture 470 may form stitches extending between tertiary suture lines 472, which are positioned closer to an end portion of the commissure tabs and farther from reinforcing elements 432 of the commissure (e.g., configured as described above with respect to reinforcing element 232 of FIG. 2) than the secondary suture lines 454.

As shown in FIGS. 4D and 4F, the suture extensions 462a and 462b may be tied together and/or knotted, or otherwise coupled together by a suitable coupling mechanism, at one or more of the loops 466, as shown by exemplary knots 468. For example, the knots 468 may be formed as described above with respect to knot 268 of FIG. 2 and/or knot 368 of FIGS. 3A and 3B.

As further shown in FIGS. 4D and 4F, which illustrate different stages of forming the additional suture loops 466, the loops may be formed by crossing the suture extensions 462a and 462b over one another and across an outer (e.g., outward-facing) surface of the support post 410 at an angle. For example, from a position of crossing and/or coupling (e.g., at the position of the knots 468), the suture extensions 462a and 462b are angled vertically relative to one another so that they each may be passed through a respective next highest (or next lowest, in examples where the sutures are formed from top to bottom of the commissure; e.g., opposite the progression shown from FIG. 4D to 4F) position on the secondary suture line 454. In this way, according to the illustrated example, the additional suture loops 466 are formed progressively upward along a height of the support strip 430/associated commissure. As noted above, in other examples, the additional suture loops 466 may be formed progressively downward along the height of the support strip 430/associated commissure.

In either of the above example approaches, the additional suture loops 466 may be formed between a topmost aperture or marking associated with the secondary suture line 454 and a bottommost aperture or marking associated with the secondary suture line 454, thereby extending along substantially the full height of the commissure/support strip 430. Alternatively, the additional suture loops 466 may only extend partway along the height of the commissure/support strip 430.

In some examples, the additional suture loops 466 may overlap (e.g., extend over) the tertiary suture 470 that forms stitches across the support post 410. In additional or alternative examples, the additional suture loops 466 may be provided over regions of the support post 410 over which the tertiary suture 470 does not pass. For example, the additional suture loops may be interspersed between stitches formed by the tertiary suture or the additional suture loops may be positioned above and/or below a region in which the tertiary suture forms stitches extending across the support post 410.

Once a final additional suture loop is formed (e.g., a suture loop formed by passing through a topmost or bottommost hole of the secondary suture line), additional coupling of the suture extensions may be performed to secure and/or tighten the additional suture loops (e.g., a surgical knot [e.g., one single knot and one double knot] and another single knot). The ends of the suture may be trimmed to remove or reduce excess suture length.

In any of the above approaches, the proposed resultant attachment configuration advantageously tightly secures the support strip of the commissure tab assembly around the support post, thereby reducing relative movement therebetween and inhibiting undesirable abrasion.

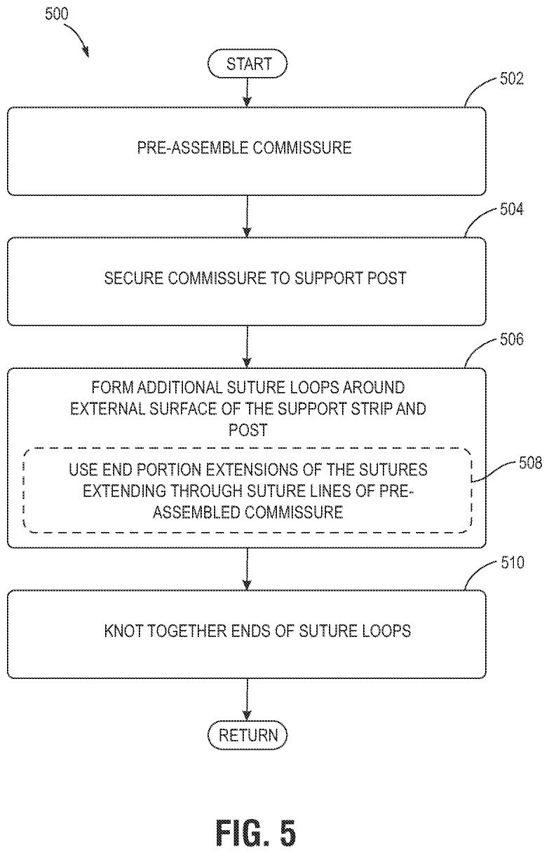

FIG. 5 is a flow chart of an example method 500 for coupling a commissure to a support structure of a frame of a prosthetic valve. For example, some or all of the operations of method 500 may be performed to assemble and secure the example commissures of FIGS. 2-4F to a support structure of a frame of a prosthetic valve.

At 502, the method includes pre-assembling the commissure. For example, the commissure may be pre-assembled as described above with respect to FIGS. 2 and 3A-3B.

At 504, the method includes securing the commissure to a support post (or other support structure of the frame). For example, the operation at 504 may include utilizing a first attachment mechanism and/or performing a first stage of attachment, which will be supplemented with a later stage of attachment as described below.

At 506, the method includes forming additional suture loops around an external surface of the support strip and post. For example, as indicated at 508, the additional suture loops may be formed using end portion extensions of the sutures extending through suture lines of the pre-assembled commissure.

At 510, the method includes knotting together (or otherwise coupling) end portions of the suture loops. For example, the end portions of the suture loops may be coupled together to form knots, such as the knots 268 described above with respect to FIG. 2, the knots 368 described above with respect to FIGS. 3A and 3B, and/or the knots 468 described above with respect to FIGS. 4D and 4F.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A method of assembling a prosthetic heart valve comprising a plurality of leaflets, the method comprising: forming a plurality of commissures with the plurality of leaflets, wherein each commissure is formed by: pairing a first commissure tab of a first leaflet with an adjacent, second commissure tab of a second leaflet, and coupling a support strip to the first and second commissure tabs via sutures extending through apertures of suture lines that are disposed on opposing sides of the support strip, the sutures forming stitches along the support strip; and, for each commissure, securing the commissure to a respective support of a frame by forming one or more suture loops around an external surface of the support strip and the respective support of the frame, wherein the one or more suture loops are formed by end portion extensions of the sutures.

Example 2. The method of any example herein, particularly example 1, further comprising, for at least one suture loop of the one or more suture loops, coupling together the end portion extensions of the sutures along the suture loop.

Example 3. The method of any example herein, particularly any one of examples 1-2, wherein the end portion extensions of the suture are respectively extended through at least a portion of the apertures of the suture lines of the support strip.

Example 4. The method of any example herein, particularly any one of examples 1-3, wherein the sutures are secondary sutures and the suture lines are secondary suture lines, and wherein each commissure is further formed by further coupling the support strip to the first and second commissure tabs via a primary suture extending through apertures of primary suture lines of the support strip, the primary suture lines being spaced from the secondary suture lines.

Example 5. The method of any example herein, particularly any one of examples 1-4, wherein the sutures are secondary sutures and the suture lines are secondary suture lines, and wherein, for each commissure, securing the commissure to the respective support of the frame further comprises extending an additional, tertiary suture through apertures of tertiary suture lines on opposing sides of the support strip and across the respective support of the frame.

Example 6. The method of any example herein, particularly example 5, wherein the tertiary suture lines are spaced from the secondary suture lines.

Example 7. The method of any example herein, particularly any one of examples 1-6, wherein, for each commissure, the end portion extensions extend from a first side of the support strip, across a surface of the respective support of the frame, to a second side of the support strip, and wherein the end portion extensions of the sutures are coupled to one another along a selected loop of the one or more suture loops, thereby forming a coupling that is disposed along the selected loop.

Example 8. The method of any example herein, particularly example 7, wherein the end portion extensions of the sutures couple to one another along a plurality of selected loops of the one or more suture loops, thereby forming a plurality of couplings that are respectively disposed along each of the plurality of selected loops.

Example 9. The method of any example herein, particularly example 8, wherein the plurality of couplings comprise a plurality of knots.

Example 10. The method of any example herein, particularly any one of examples 8-9, wherein the end portion extensions of the sutures couple to one another along each of the one or more suture loops, and wherein the plurality of couplings are respectively disposed along each of the one or more suture loops.

Example 11. The method of any example herein, particularly any one of examples 1-10, wherein the apertures of the suture lines are positioned at a first region of an interior side of the support of the frame, and wherein the one or more suture loops extend from a first region of the interior side of the support, around a first lateral side of the support, across an external side of the support, around a second lateral side of the support, and to a second region of the interior side of the support, wherein the interior side of the support is opposite of the external side of the support and the first and second lateral sides of the support are opposite of one another and extend between the interior side of the support and the external side of the support.

Example 12. The method of any example herein, particularly any one of examples 1-11, wherein the one or more suture loops extend more than 270 degrees around the support of the frame.

Example 13. The method of any example herein, particularly any one of examples 1-12, wherein the sutures forming stitches along the support strip extend perpendicularly to the one or more suture loops.

Example 14. The method of any example herein, particularly any one of examples 1-13, wherein the suture lines extend at an angle relative to the one or more suture loops, the angle being between 80 degrees and 100 degrees.

Example 15. The method of any example herein, particularly any one of examples 1-14, wherein the opposing sides of the support strip include a first side and a second side, the method further comprising folding the first side of the support strip around a first reinforcing element and folding the second side of the support strip around a second reinforcing element, wherein the sutures extending through apertures of the suture line that is disposed on the first side of the support strip extend through multiple layers of the first side of the support strip adjacent the first reinforcing element, and wherein the sutures extending through apertures of the suture line that is disposed on the second side of the support strip extend through multiple layers of the second side of the support strip adjacent the second reinforcing element.

Example 16. The method of any example herein, particularly example 15, wherein the first reinforcing element and the second reinforcing element are two portions of a single reinforcing member that extends along outer surfaces of each of the first and second commissure tabs.

Example 17. The method of any example herein, particularly example 15, wherein the first reinforcing element and the second reinforcing element are discrete elements of a reinforcing member that are spatially separated from one another.

Example 18. A prosthetic heart valve comprising: an annular frame comprising a plurality of commissure support portions; and a plurality of leaflets, each leaflet having a commissure tab that is coupled to an adjacent commissure tab of another leaflet via a support strip to form a commissure of an associated commissure tab pair, wherein, for each commissure tab pair: sutures extend through apertures of suture lines that are disposed on opposing sides of the support strip, the sutures forming stitches along the support strip coupling the support strip to each commissure tab of the commissure tab pair, and wherein each commissure is secured to a corresponding respective commissure support portion of the plurality of commissure support portions by one or more suture loops that are formed by end portion extensions of the sutures that extend around an external surface of the support strip and the respective commissure support portion of the annular frame.

Example 19. The prosthetic heart valve of any example herein, particularly example 18, wherein, for each commissure tab pair, the end portion extensions extend from a first side of the support strip, across a surface of the respective commissure support portion, to a second side of the support strip, and wherein the end portion extensions of the sutures are coupled to one another along a selected loop of the one or more suture loops, thereby forming a coupling that is disposed along the selected loop.

Example 20. The prosthetic heart valve of any example herein, particularly example 19, wherein the end portion extensions of the sutures couple to one another along a plurality of selected loops of the one or more suture loops, thereby forming a plurality of couplings that are respectively disposed along each of the plurality of selected loops.

Example 21. The prosthetic heart valve of any example herein, particularly example 20, wherein the end portion extensions of the sutures couple to one another along each of the one or more suture loops, thereby forming a plurality of couplings that are respectively disposed along each of the one or more suture loops.

Example 22. The prosthetic heart valve of any example herein, particularly any one of examples 20-21, wherein the plurality of couplings comprise a plurality of knots.

Example 23. The prosthetic heart valve of any example herein, particularly any one of examples 18-22, wherein the end portion extensions of the suture respectively extend through at least a portion of the apertures of the suture lines of the support strip.

Example 24. The prosthetic heart valve of any example herein, particularly any one of examples 18-23, wherein the sutures are secondary sutures and the suture lines are secondary suture lines, wherein the support strip couples to the first and second commissure tabs via a primary suture that extends through apertures of primary suture lines of the support strip, and wherein the primary suture lines are spaced from the secondary suture lines.

Example 25. The prosthetic heart valve of any example herein, particularly any one of examples 18-24, wherein the sutures are secondary sutures and the suture lines are secondary suture lines, and wherein, for each commissure, an additional, tertiary suture extends through apertures of tertiary suture lines on opposing sides of the support strip and across the respective commissure support portion of the frame to secure the commissure to the respective commissure support portion of the frame.

Example 26. The prosthetic heart valve of any example herein, particularly example 25, wherein the tertiary suture lines are spaced from the secondary suture lines.

Example 27. The prosthetic heart valve of any example herein, particularly any one of examples 18-26, wherein the apertures of the suture lines are positioned at a first region of an interior side of the commissure support portion, and wherein the one or more suture loops extend from a first region of the interior side of the commissure support portion, around a first lateral side of the commissure support portion, across an external side of the commissure support portion, around a second lateral side of the commissure support portion, and to a second region of the interior side of the commissure support portion, wherein the interior side of the commissure support portion is opposite of the external side of the commissure support portion and the first and second lateral sides of the commissure support portion are opposite of one another and extend between the interior side of the commissure support portion and the external side of the commissure support portion.

Example 28. The prosthetic heart valve of any example herein, particularly any one of examples 18-27, wherein the one or more suture loops extend more than 270 degrees around the commissure support portion.

Example 29. The prosthetic heart valve of any example herein, particularly any one of examples 18-28, wherein the sutures forming stitches along the support strip extend perpendicularly to the one or more suture loops.

Example 30. The prosthetic heart valve of any example herein, particularly any one of examples 18-29, wherein the suture lines extend at an angle relative to the one or more suture loops, the angle being between 80 degrees and 100 degrees.

Example 31. The prosthetic heart valve of any example herein, particularly any one of examples 18-30, further comprising a first reinforcing element and a second reinforcing element, wherein the opposing sides of the support strip include a first side and a second side, wherein the first side of the support strip is folded around the first reinforcing element and the second side of the support strip is folded around the second reinforcing element, wherein the sutures extending through apertures of the suture line that is disposed on the first side of the support strip extend through multiple layers of the first side of the support strip adjacent the first reinforcing element, and wherein the sutures extending through apertures of the suture line that is disposed on the second side of the support strip extend through multiple layers of the second side of the support strip adjacent the second reinforcing element.

Example 32. The prosthetic heart valve of any example herein, particularly example 31, wherein the first reinforcing element and the second reinforcing element are two portions of a single reinforcing member that extends along outer surfaces of each of the first and second commissure tabs.

Example 33. The prosthetic heart valve of any example herein, particularly example 31, wherein the first reinforcing element and the second reinforcing element are discrete elements of a reinforcing member that are spatially separated from one another.

Example 34. A method of assembling a prosthetic heart valve comprising a plurality of leaflets, the method comprising: forming a plurality of commissures with the plurality of leaflets, wherein each commissure is formed by: pairing a first commissure tab of a first leaflet with an adjacent, second commissure tab of a second leaflet, coupling a support strip to the first and second commissure tabs via primary sutures extending through the support strip and the first and second commissure tabs to form stitches along primary suture lines of the support strip, and coupling end portions of the support strip to the first and second commissure tabs, respectively, via secondary sutures extending through the support strip and the first and second commissure tabs, respectively, to form stitches along respective secondary suture lines of the support strip; and, for each commissure, securing the commissure to a respective commissure support portion of an annular frame of the prosthetic heart valve by one or more suture loops that are formed by end portion extensions of the secondary sutures that extend around an external surface of the support strip and the respective commissure support portion of the annular frame.

Example 35. The method of any example herein, particularly example 34, further comprising, for at least one suture loop of the one or more suture loops, coupling together the end portion extensions of the sutures along the suture loop.

Example 36. The method of any example herein, particularly any one of examples 34-35, wherein the end portion extensions of the suture are respectively extended through apertures of the secondary suture lines of the support strip.

Example 37. The method of any example herein, particularly any one of examples 34-36, wherein, for each commissure, securing the commissure to the respective support of the frame further comprises extending an additional, tertiary suture through apertures of tertiary suture lines on opposing sides of the support strip and across the respective commissure support portion of the frame.

Example 38. The method of any example herein, particularly any one of examples 34-37, wherein, for each commissure, the end portion extensions extend from a first side of the support strip, across a surface of the respective support of the frame, to a second side of the support strip, and wherein the end portion extensions of the sutures are coupled to one another along a selected loop of the one or more suture loops, thereby forming a coupling that is disposed along the selected loop.

Example 39. The method of any example herein, particularly example 38, wherein the end portion extensions of the sutures couple to one another along a plurality of selected loops of the one or more suture loops, thereby forming a plurality of couplings that are respectively disposed along each of the plurality of selected loops.

Example 40. The method of any example herein, particularly example 39, wherein the plurality of couplings comprise a plurality of knots.

Example 41. The method of any example herein, particularly any one of examples 39-40, wherein the end portion extensions of the sutures couple to one another along each of the one or more suture loops, and wherein the plurality of couplings are respectively disposed along each of the one or more suture loops.

Example 42. The method of any example herein, particularly any one of examples 34-41, wherein the apertures of the suture lines are positioned at a first region of an interior side of the commissure support portion, and wherein the one or more suture loops extend from a first region of the interior side of the commissure support portion, around a first lateral side of the commissure support portion, across an external side of the commissure support portion, around a second lateral side of the commissure support portion, and to a second region of the interior side of the commissure support portion, wherein the interior side of the commissure support portion is opposite of the external side of the commissure support portion and the first and second lateral sides of the commissure support portion are opposite of one another and extend between the interior side of the commissure support portion and the external side of the commissure support portion.

Example 43. The method of any example herein, particularly any one of examples 34-42, wherein the one or more suture loops extend more than 270 degrees around the commissure support portion.

Example 44. The method of any example herein, particularly any one of examples 34-43, wherein the sutures forming stitches along the support strip extend perpendicularly to the one or more suture loops.

Example 45. The method of any example herein, particularly any one of examples 34-44, wherein the suture lines extend at an angle relative to the one or more suture loops, the angle being between 80 degrees and 100 degrees.

Example 46. The method of any example herein, particularly any one of examples 34-45, wherein the respective secondary suture lines comprises a first suture line positioned on a first side of the support strip and a second suture line positioned on a second side of the support strip, the method further comprising folding the first side of the support strip around a first reinforcing element and folding the second side of the support strip around a second reinforcing element, wherein the secondary sutures forming stitches along the first suture line extend through multiple layers of the first side of the support strip adjacent the first reinforcing element, and wherein the secondary sutures forming stitches along the second suture line extend through multiple layers of the second side of the support strip adjacent the second reinforcing element.

Example 47. The method of any example herein, particularly example 46, wherein the first reinforcing element and the second reinforcing element are two portions of a single reinforcing member that extends along outer surfaces of each of the first and second commissure tabs.

Example 48. The method of any example herein, particularly example 46, wherein the first reinforcing element and the second reinforcing element are discrete elements of a reinforcing member that are spatially separated from one another.

Example 49. A prosthetic heart valve comprising: an annular frame comprising a plurality of commissure support portions; and a plurality of leaflets, each leaflet having a commissure tab that is coupled to an adjacent commissure tab of another leaflet via a support strip to form a commissure of an associated commissure tab pair, wherein, for each commissure tab pair: sutures extend through apertures of suture lines that are disposed on opposing sides of the support strip, the sutures forming stitches along the support strip coupling the support strip to each commissure tab of the commissure tab pair, and wherein each commissure is secured to a corresponding respective commissure support portion of the plurality of commissure support portions by one or more suture loops that are formed by end portion extensions of the sutures that extend around an external surface of the support strip and the respective commissure support portion of the annular frame, and wherein, for each leaflet: a cusp edge portion of the leaflet extending from the respective commissure tab of the leaflet is coupled to an inner skirt and around adjacent struts of the annular frame.

Example 50. The prosthetic heart valve of any example herein, particularly example 49, wherein, for each commissure tab pair, the end portion extensions extend from a first side of the support strip, across a surface of the respective commissure support portion, to a second side of the support strip, and wherein the end portion extensions of the sutures are coupled to one another along a selected loop of the one or more suture loops, thereby forming a coupling that is disposed along the selected loop.

Example 51. The prosthetic heart valve of any example herein, particularly example 50, wherein the end portion extensions of the sutures couple to one another along a plurality of selected loops of the one or more suture loops, thereby forming a plurality of couplings that are respectively disposed along each of the plurality of selected loops.

Example 52. The prosthetic heart valve of any example herein, particularly example 51, wherein the end portion extensions of the sutures couple to one another along each of the one or more suture loops, thereby forming a plurality of couplings that are respectively disposed along each of the one or more suture loops.

Example 53. The prosthetic heart valve of any example herein, particularly any one of examples 51-52, wherein the plurality of couplings comprise a plurality of knots.

Example 54. The prosthetic heart valve of any example herein, particularly any one of examples 49-53, wherein the end portion extensions of the suture respectively extend through at least a portion of the apertures of the suture lines of the support strip.

Example 55. The prosthetic heart valve of any example herein, particularly any one of examples 49-54, wherein the sutures are secondary sutures and the suture lines are secondary suture lines, wherein the support strip couples to the first and second commissure tabs via a primary suture that extends through apertures of primary suture lines of the support strip, and wherein the primary suture lines are spaced from the secondary suture lines.

Example 56. The prosthetic heart valve of any example herein, particularly any one of examples 49-55, wherein the sutures are secondary sutures and the suture lines are secondary suture lines, and wherein, for each commissure, an additional, tertiary suture extends through apertures of tertiary suture lines on opposing sides of the support strip and across the respective commissure support portion of the frame to secure the commissure to the respective commissure support portion of the frame.

Example 57. The prosthetic heart valve of any example herein, particularly example 56, wherein the tertiary suture lines are spaced from the secondary suture lines.

Example 58. The prosthetic heart valve of any example herein, particularly any one of examples 49-57, wherein the apertures of the suture lines are positioned at a first region of an interior side of the commissure support portion, and wherein the one or more suture loops extend from a first region of the interior side of the commissure support portion, around a first lateral side of the commissure support portion, across an external side of the commissure support portion, around a second lateral side of the commissure support portion, and to a second region of the interior side of the commissure support portion, wherein the interior side of the commissure support portion is opposite of the external side of the commissure support portion and the first and second lateral sides of the commissure support portion are opposite of one another and extend between the interior side of the commissure support portion and the external side of the commissure support portion.

Example 59. The prosthetic heart valve of any example herein, particularly any one of examples 49-58, wherein the one or more suture loops extend more than 270 degrees around the commissure support portion.

Example 60. The prosthetic heart valve of any example herein, particularly any one of examples 49-59, wherein the sutures forming stitches along the support strip extend perpendicularly to the one or more suture loops.

Example 61. The prosthetic heart valve of any example herein, particularly any one of examples 49-60, wherein the suture lines extend at an angle relative to the one or more suture loops, the angle being between 80 degrees and 100 degrees.

Example 62. The prosthetic heart valve of any example herein, particularly any one of examples 49-61, further comprising a first reinforcing element and a second reinforcing element, wherein the opposing sides of the support strip include a first side and a second side, wherein the first side of the support strip is folded around the first reinforcing element and the second side of the support strip is folded around the second reinforcing element, wherein the sutures extending through apertures of the suture line that is disposed on the first side of the support strip extend through multiple layers of the first side of the support strip adjacent the first reinforcing element, and wherein the sutures extending through apertures of the suture line that is disposed on the second side of the support strip extend through multiple layers of the second side of the support strip adjacent the second reinforcing element.

Example 63. The prosthetic heart valve of any example herein, particularly example 62, wherein the first reinforcing element and the second reinforcing element are two portions of a single reinforcing member that extends along outer surfaces of each of the first and second commissure tabs.

Example 64. The prosthetic heart valve of any example herein, particularly example 62, wherein the first reinforcing element and the second reinforcing element are discrete elements of a reinforcing member that are spatially separated from one another.

Example 65. A prosthetic heart valve comprising: an annular frame comprising a plurality of commissure support portions; and a plurality of leaflets, each leaflet having a commissure tab that is coupled to an adjacent commissure tab of another leaflet via a support strip to form a commissure of an associated commissure tab pair, wherein, for each commissure tab pair: a primary suture passes through, in order or in reverse order, a first side of the support strip, a first commissure tab of the commissure tab pair, a second commissure tab of the commissure tab pair, and a second side of the support strip, and secondary sutures pass through the first and second sides of the support strip, respectively, and wherein one or more suture loops secure each commissure to a corresponding respective commissure support portion of the plurality of commissure support portions, wherein end portion extensions of the secondary sutures extend around an external surface of the support strip and the respective commissure support portion of the annular frame to form the one or more suture loops.

Example 66. The prosthetic heart valve of any example herein, particularly example 65, wherein the end portion extensions of the sutures are coupled to one another along a selected loop of the one or more suture loops, thereby forming a coupling that is disposed along the selected loop.

Example 67. The prosthetic heart valve of any example herein, particularly example 66, wherein the end portion extensions of the sutures couple to one another along a plurality of selected loops of the one or more suture loops, thereby forming a plurality of couplings that are respectively disposed along each of the plurality of selected loops.

Example 68. The prosthetic heart valve of any example herein, particularly example 67, wherein the end portion extensions of the sutures couple to one another along each of the one or more suture loops, thereby forming a plurality of couplings that are respectively disposed along each of the one or more suture loops.

Example 69. The prosthetic heart valve of any example herein, particularly any one of examples 65-68, wherein, for each commissure, an additional, tertiary suture extends through apertures of tertiary suture lines on opposing sides of the support strip and across the respective commissure support portion of the frame to secure the commissure to the respective commissure support portion of the frame.

Example 70. The prosthetic heart valve of any example herein, particularly example 69, wherein the tertiary suture lines are spaced from the secondary suture lines.

Example 71. The prosthetic heart valve of any example herein, particularly any one of examples 65-70, wherein the one or more suture loops extend more than 270 degrees around the commissure support portion.

Example 72. The prosthetic heart valve of any example herein, particularly any one of examples 65-71, wherein the suture lines extend at an angle relative to the one or more suture loops, the angle being between 80 degrees and 100 degrees.

Example 73. The prosthetic heart valve of any example herein, particularly any one of examples 65-72, further comprising a first reinforcing element and a second reinforcing element, wherein the first side of the support strip is folded around the first reinforcing element and the second side of the support strip is folded around the second reinforcing element, wherein the sutures extending through apertures of the suture line that is disposed on the first side of the support strip extend through multiple layers of the first side of the support strip adjacent the first reinforcing element, and wherein the sutures extending through apertures of the suture line that is disposed on the second side of the support strip extend through multiple layers of the second side of the support strip adjacent the second reinforcing element.

Example 74. The prosthetic heart valve of any example herein, particularly example 73, wherein the first reinforcing element and the second reinforcing element are two portions of a single reinforcing member that extends along outer surfaces of each of the first and second commissure tabs.

Example 75. The prosthetic heart valve of any example herein, particularly example 73, wherein the first reinforcing element and the second reinforcing element are discrete elements of a reinforcing member that are spatially separated from one another.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of assembling a prosthetic heart valve comprising a plurality of leaflets, the method comprising:
forming a plurality of commissures with the plurality of leaflets, wherein each commissure is formed by:
pairing a first commissure tab of a first leaflet with an adjacent, second commissure tab of a second leaflet, and
coupling a support strip to the first and second commissure tabs via sutures extending through apertures of suture lines that are disposed on opposing sides of the support strip, the sutures forming stitches along the support strip; and
for each commissure, securing the commissure to a respective support of a frame by forming one or more suture loops around an external surface of the support strip and the respective support of the frame, wherein the one or more suture loops are formed by end portion extensions of the sutures.

2. The method of claim 1, wherein the sutures are secondary sutures and the suture lines are secondary suture lines, and wherein each commissure is further formed by further coupling the support strip to the first and second commissure tabs via a primary suture extending through apertures of primary suture lines of the support strip, the primary suture lines being spaced from the secondary suture lines.

3. The method of claim 1, wherein the sutures are secondary sutures and the suture lines are secondary suture lines, and wherein, for each commissure, securing the commissure to the respective support of the frame further comprises extending an additional, tertiary suture through apertures of tertiary suture lines on opposing sides of the support strip and across the respective support of the frame.

4. The method of claim 1, wherein, for each commissure, the end portion extensions extend from a first side of the support strip, across a surface of the respective support of the frame, to a second side of the support strip, and wherein the end portion extensions of the sutures are coupled to one another along a selected loop of the one or more suture loops, thereby forming a coupling that is disposed along the selected loop.

5. The method of claim 4, wherein the end portion extensions of the sutures couple to one another along a plurality of selected loops of the one or more suture loops, thereby forming a plurality of couplings that are respectively disposed along each of the plurality of selected loops.

6. The method of claim 1, wherein the apertures of the suture lines are positioned at a first region of an interior side of the support of the frame, and wherein the one or more suture loops extend from a first region of the interior side of the support, around a first lateral side of the support, across an external side of the support, around a second lateral side of the support, and to a second region of the interior side of the support, wherein the interior side of the support is opposite of the external side of the support and the first and second lateral sides of the support are opposite of one another and extend between the interior side of the support and the external side of the support.

7. The method of claim 1, wherein the opposing sides of the support strip include a first side and a second side, the method further comprising folding the first side of the support strip around a first reinforcing element and folding the second side of the support strip around a second reinforcing element, wherein the sutures extending through apertures of the suture line that is disposed on the first side of the support strip extend through multiple layers of the first side of the support strip adjacent the first reinforcing element, and wherein the sutures extending through apertures of the suture line that is disposed on the second side of the support strip extend through multiple layers of the second side of the support strip adjacent the second reinforcing element.

8. A prosthetic heart valve comprising:
an annular frame comprising a plurality of commissure support portions; and
a plurality of leaflets, each leaflet having a commissure tab that is coupled to an adjacent commissure tab of another leaflet via a support strip to form a commissure of an associated commissure tab pair,
wherein, for each commissure tab pair:
sutures extend through apertures of suture lines that are disposed on opposing sides of the support strip, the sutures forming stitches along the support strip coupling the support strip to each commissure tab of the commissure tab pair, and
wherein each commissure is secured to a corresponding respective commissure support portion of the plurality of commissure support portions by one or more suture loops that are formed by end portion extensions of the sutures that extend around an external surface of the support strip and the respective commissure support portion of the annular frame.

9. The prosthetic heart valve of claim 8, wherein, for each commissure tab pair, the end portion extensions extend from a first side of the support strip, across a surface of the respective commissure support portion, to a second side of the support strip, and wherein the end portion extensions of the sutures are coupled to one another along a selected loop of the one or more suture loops, thereby forming a coupling that is disposed along the selected loop.

10. The prosthetic heart valve of claim 9, wherein the end portion extensions of the sutures couple to one another along a plurality of selected loops of the one or more suture loops, thereby forming a plurality of couplings that are respectively disposed along each of the plurality of selected loops.

11. The prosthetic heart valve of claim 8, wherein the sutures are secondary sutures and the suture lines are secondary suture lines, wherein the support strip couples to the commissures of the commissure tab pair via a primary suture that extends through apertures of primary suture lines of the support strip, and wherein the primary suture lines are spaced from the secondary suture lines.

12. The prosthetic heart valve of claim 8, wherein the sutures are secondary sutures and the suture lines are secondary suture lines, and wherein, for each commissure, an additional, tertiary suture extends through apertures of tertiary suture lines on opposing sides of the support strip and across the respective commissure support portion of the frame to secure the commissure to the respective commissure support portion of the frame.

13. The prosthetic heart valve of claim 8, wherein the apertures of the suture lines are positioned at a first region of an interior side of the commissure support portion, and wherein the one or more suture loops extend from a first region of the interior side of the commissure support portion, around a first lateral side of the commissure support portion, across an external side of the commissure support portion, around a second lateral side of the commissure support portion, and to a second region of the interior side of the commissure support portion, wherein the interior side of the commissure support portion is opposite of the external side of the commissure support portion and the first and second lateral sides of the commissure support portion are opposite of one another and extend between the interior side of the commissure support portion and the external side of the commissure support portion.

14. The prosthetic heart valve of claim 8, further comprising a first reinforcing element and a second reinforcing element, wherein the opposing sides of the support strip include a first side and a second side, wherein the first side of the support strip is folded around the first reinforcing element and the second side of the support strip is folded around the second reinforcing element, wherein the sutures extending through apertures of the suture line that is disposed on the first side of the support strip extend through multiple layers of the first side of the support strip adjacent the first reinforcing element, and wherein the sutures extending through apertures of the suture line that is disposed on the second side of the support strip extend through multiple layers of the second side of the support strip adjacent the second reinforcing element.

15. The prosthetic heart valve of claim 14, wherein the first reinforcing element and the second reinforcing element are two portions of a single reinforcing member that extends along outer surfaces of each of the commissure tabs of the commissure tab pair.

16. The prosthetic heart valve of claim 14, wherein the first reinforcing element and the second reinforcing element are discrete elements of a reinforcing member that are spatially separated from one another.

17. A method of assembling a prosthetic heart valve comprising a plurality of leaflets, the method comprising:
forming a plurality of commissures with the plurality of leaflets, wherein each commissure is formed by:
pairing a first commissure tab of a first leaflet with an adjacent, second commissure tab of a second leaflet,
coupling a support strip to the first and second commissure tabs via primary sutures extending through the support strip and the first and second commissure tabs to form stitches along primary suture lines of the support strip, and
coupling end portions of the support strip to the first and second commissure tabs, respectively, via secondary sutures extending through the support strip and the first and second commissure tabs, respectively, to form stitches along respective secondary suture lines of the support strip; and
for each commissure, securing the commissure to a respective commissure support portion of an annular frame of the prosthetic heart valve by one or more suture loops that are formed by end portion extensions of the secondary sutures that extend around an external surface of the support strip and the respective commissure support portion of the annular frame.

18. The method of claim 17, further comprising, for at least one suture loop of the one or more suture loops, coupling together the end portion extensions of the sutures along the suture loop.

19. The method of claim 17, wherein the end portion extensions of the suture are respectively extended through apertures of the secondary suture lines of the support strip.

20. The method of claim 17, wherein, for each commissure, securing the commissure to the respective support of the frame further comprises extending an additional, tertiary suture through apertures of tertiary suture lines on opposing sides of the support strip and across the respective commissure support portion of the frame.
